(19) United States
(12) Patent Application Publication
(10) Pub. No.: US 2007/0218465 A1
(43) Pub. Date: Sep. 20, 2007

(54) Therapeutic Applications of Aminolevulinate Synthase

(75) Inventors: Gregory A. Hunter, Tampa, FL (US); Gloria C. Ferreira, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 11/427,604

(22) Filed: Jun. 29, 2006

Related U.S. Application Data
(60) Provisional application No. 60/695,172, filed on Jun. 29, 2005.

(51) Int. Cl. C40B 30/06; C40B 40/08; A61K 31/409; A61K 31/195

(52) U.S. Cl. 435/6; 435/91.2; 514/410; 514/561

(57) ABSTRACT

Systems and methods for increasing protoporphyrin IX accumulation in a target cell population using aminolevulinate synthase variants. Aminolevulinic acid-mediated photodynamic therapy is a promising approach to treating dysplasic disorders such as cancer and atherosclerosis, but is limited by the lack of a means to deliver optimal quantities of aminolevulinic acid selectively to the target cells, and thereby ensure the best therapeutic response. The disclosed invention provides a means for enhancing the natural production of aminolevulinic acid selectively within target cells to levels predetermined to give an optimal therapeutic response, and is expected to lead to increased efficacy of treatment, possibly broadening the scope of diseases treatable by photodynamic therapy considerably. The disclosed invention is also amenable to patient specific therapy, meaning that a patient's target cells could be used to screen for the aminolevulinic acid delivery system most appropriate for the patient's needs.

### CROSS REFERENCE TO RELATED APPLICATIONS

This application claims priority to currently pending U.S. Provisional Patent Application 60/695,172, entitled, “Therapeutic Applications of Aminolevulinate Synthase”, filed Jun. 29, 2005, the contents of which are herein incorporated by reference.

### STATEMENT OF GOVERNMENT INTEREST

This invention was made with Government support under Grant No. DK63191 awarded by the National Institute of Health. The Government has certain rights in the invention.

### FIELD OF INVENTION

This invention relates generally to the field of medicine. More specifically, this invention relates to the treatment of diseases characterized by hyperproliferative, cancerous or other abnormal cells using photodynamic therapy.

### BACKGROUND OF THE INVENTION

Photodynamic therapy (PDT) is a treatment that is based upon the differential uptake by cancerous or other target cells of photosensitizing agents, followed by irradiation of the cells to cause a photochemical reaction. Photodynamic therapy involves the administration of a photosensitizing agent to a subject, including administration of a precursor of a photosensitizing agent such as aminolevulinic acid (ALA), and subsequent irradiation with light of the target cells or tissue of the subject. The photosensitizing agent preferentially accumulates in the target cells. These target cells or tissues are characterized by rapid proliferation or growth relative to the surrounding cells or tissues in the target environment. The target cells may be more rapidly proliferating because they are malignant or non-malignant, of infective agent origin, e.g. viral, bacteria, parasite or fungal origin or not of infective agent origin; are normally hyperproliferative, such as the endometrium of pre-menopausal women, or are abnormally hyperproliferative, such as cells infected with an infective agent. Although not intending to be bound by any particularly theory, it has been proposed that following administration of ALA, as a result of their more rapid proliferation, the target cells or tissue contain relatively greater concentrations of light sensitive porphyrins and thus are more sensitive to light. The photochemical reaction is believed to generate chemically disruptive species, such as singlet oxygen. These disruptive species in turn injure the cells through reaction with cell parts, such as cellular and nuclear membranes. Photodynamic therapy has been used successfully for treating several types of cancer cells.

Tobacco smoking is the leading preventable cause of death in the United States. A well-defined correlation exists between exposure to tobacco smoke and several cancers, including lung, esophageal, bladder, larynx, and oral cancers. Exposure to tobacco smoke also promotes coronary artery atherosclerosis, and is the main cause of abdominal aortic aneurysms. Photodynamic therapy represents a growing area of clinical applications that show great promise in the treatment of these tobacco related diseases, but complete remission of disease using photodynamic therapy is often difficult to achieve. One way to enhance the efficacy of photodynamic therapy regimes would be to provide a means for the selective accumulation of photosensitizing agent within the target tissues to levels not currently possible. The painful side-effect of photosensitivity necessitates that these increased concentrations be delivered in as localized a manner as feasible. If high-level selective photosensitizer accumulation were possible it would open the door for a much larger number of patients suffering from tobacco related diseases to be effectively treated with photodynamic therapy. Furthermore, the instant methodologies offer great promise for enhancing the effectivesness of photodynamic therapy in other applications for treating diseases such as those characterized by a more rapid proliferation of target cells or tissues. The present invention offers one possible way to achieve high-level selective photosensitizer accumulation. By identifying and delivering to target cells the enzymatic capacity to hyper-stimulate protoporphyrin IX production, the selective photosensitizer accumulation could conceivably be elevated to levels not previously possible, with resulting increases in efficacy of treatment and expansion of treatable populations.

PDT protocols for the treatment of disease typically involve the administration of a photosensitizer from about 15 minutes to about 2 days prior to the application of light. This allows the photosensitizer time to accumulate in the target disease tissue, while being cleared from normal tissue. Concerns in the use of the treatment include its safety and effectiveness.

PDT can have associated side-effects. For example, at the target site, PDT has been associated with the development of inflammation with edema and pain, and even necrosis with scarring. With systemically delivered photosensitizers formulated in either aqueous or organic solvents, or in liposomal formulations, the side effects can include headaches, nausea, and fever, as well as skin photosensitivity. Moreover, the greater the dosage of photosensitizers used, the greater the risk of these, and potentially other, side effects. However, if too little photosensitizer is used in the treatment, then there is a greater risk of having only a partial response to treatment or recurrence of disease. Therefore, what is needed is a method to allow the more selective accumulation of photosensitzer in the target cells relative to surrounding tissue. Such a selective accumulation would enable the effective treatment of the diseased target area while limiting collateral effects.

### SUMMARY OF INVENTION

The invention provides a means whereby optimal concentrations of the photosensitizing agent precursor aminolevulinic acid can be selectively achieved in target cell populations, which provides more effective therapeutic treatments for cancers and heart diseases. Specifically, the invention involves a procedure for the creation, identification, and use of 5-aminolevulinate synthase variants conferring maximal photosensitivity upon a given cell population. Initially, genetically diverse libraries of 5-aminolevulinate synthase variants are constructed using a variety of directed evolution techniques (10⁶-10⁹ different variants). Overexpression of 5-aminolevulinate synthase activity in mammals stimulates accumulation of protoporphyrin IX, which confers photosensitivity and is fluorescent. The libraries are expressed in mammalian cell lines for screening using flow cytometry and fluorescent activated cell sorting. The cell line chosen for screening aminolevulinate synthase libraries mimics as closely as possible the cell population targeted for eradication by photodynamic therapy. Ideally, screening is conducted using the patient's diseased cells. In this way possible differences amongst various cell phenotypes that could negatively affect optimal expression of enzyme activity are accounted for during the screening process. The variant stimulating maximal protoporphyrin IX production (i.e., conferring maximal photosensitivity) is identified and isolated. The 5-aminolevulinate synthase coding region is then isolated for targeted delivery to the diseased cells and phototherapy. Delivery could be in either protein or nucleic acid form. Individuals with targetable cancers or heart diseases would be one group appropriate for this technology.

Tobacco related diseases such as cancer and atherosclerosis often respond favorably to photodynamic therapy with 5-aminolevulinic acid. A major drawback to this therapeutic approach is the lack of a mechanism to selectively concentrate the photosensitizing agent within the target cells and thereby achieve higher intracellular concentrations, which would allow a more complete eradication of diseased cells. The present invention provides such a mechanism. Aspects of the invention include the construction and isolation of variants of 5-aminolevulinate synthase that stimulate optimal production of the photosensitizing agent protoporphyrin IX specifically in the cells to be targeted. The purified enzyme can then be biochemically targeted to the diseased cells, where it stimulates accumulation of photosensitizing agent to levels not previous possible. This results in more effective treatments. Furthermore, the incidence of complete remissions may be found to increase dramatically.

The invention is based, in part, on the discovery that a rate limiting step in the accumulation of ALA in the target cell is the ability of aminolevulinate synthase to act upon the precursor molecules. By increasing the aminolevulinate synthase, optimal production of the photosensitizing agent protoporphyrin IX can be achieved. Furthermore, by screening for optimal activity in the target cell type, the ALAS variant may cause reduced collateral damage to the extent that it displays correspondingly lower activity in the cell types of the tissue surrounding the target cells.

According to one aspect of the invention there is provided methods of producing a 5-aminolevulinate synthase (ALAS) variants having enhanced protoporphyrin IX accumulation in a target cell population by (a) generating a cDNA library of ALAS variants; (b) providing a target cell population; (c) introducing in vitro the ALAS variants into the target cells; (d) screening the target cells for protoporphyrin IX accumulation; and (e) identifying the ALAS variants having enhanced protoporphyrin IX accumulation according to the results of screening. In these methods the cDNA library of ALAS variants can be generated using techniques such as error-prone PCR, DNA shuffling, DNA family shuffling and synthetic shuffling. The transformed target cells can be screened for protoporphyrin IX accumulation by fluorescent activated cell sorting. In an advantageous aspect the target cell population is a non-small cell lung cancer cell line. It is further contemplated that the target cell population can be a cell population of interest removed from a patient undergoing patient-specific therapy. In this aspect, the patient's cells are used to screen for ALAS variants having enhanced protoporphyrin IX accumulation in the patient's cell of interest, thus specifically tailoring the treatment to the patient's disease. Patient benefiting from the treatment will include cancer patients, such as those having non-small cell lung cancer.

The constructs in the cDNA library of ALAS variants can me modified to encode ALAS variant fusion proteins having a cellular uptake sequence. By incorporating the cellular uptake sequence, the variants can be adapted to display enhanced uptake into the target cell. An advantageous cellular uptake sequence is a sequence derived from epidermal growth factor (EGF). This sequence binds the EGF receptor and facilitates passage through the cell surface. Other cellular uptake sequences can be used in generating the fusion protein as are known in the art.

In additional embodiments of the invention there is provided methods of producing a 5-aminolevulinate synthase (ALAS) variant having enhanced activity in a target cell population. The methods include the steps of (a) generating a cDNA library of ALAS variants; (b) providing a target cell population; (c) introducing in vitro the ALAS variants into the target cells; (d) screening the target cells for an indicia of ALAS activity; and (e) identifying the ALAS variants having enhanced ALAS activity according to the results of screening. It is contemplated that the target cell population can be a cell population of interest removed from a patient undergoing patient-specific therapy. In this manner, the patient's cells are used to screen for ALAS variants having enhanced ALAS activity in the patient's cell of interest.

In yet additional embodiments of the invention there is provided methods for treating a target tissue in a subject with photodynamic therapy. The methods can include the steps of (a) administering to the subject an effective amount of 5-aminolevulinate synthase (ALAS), or an analog or variant thereof, or a nucleic acid encoding a 5-aminolevulinate synthase, or an analog or variant thereof, and (b) irradiating the target tissue of the subject using radiation in an amount and of a wavelength effective to activate the 5-aminolevulinic acid variant, thereby treating a target tissue in the subject with photodynamic therapy. In certain embodiments the step of administering and the step of irradiating are separated by at least about 2 hours. In other embodiments the step of administering and the step of irradiating are separated by about 2 hours to about 48 hours. In still other embodiments the step of administering and the step of irradiating are separated by about 2 hours to about 24 hours. The methods can further include the step of administering to the subject an effective amount of a porphyrin precursor such as 5-aminolevulinic acid. The compound to be administered in the subject's treatment can be selected based upon screening assay data demonstrating enhanced in vitro protoporphyrin IX accumulation in the target cell type following administration of the compound. The methods of treating a target tissue with photodynamic therapy may further include the steps of (a) providing a target cell population; (b) introducing in vitro one or more ALAS variants into the target cells; (c) screening the target cells for protoporphyrin IX accumulation; and (d) identifying the ALAS variants having enhanced protoporphyrin IX accumulation according to the results of screening, wherein one or more ALAS variants identified will be used for administration to the patient. The target cell population used for screening can be a cell population derived from the target tissue of the subject to be treated.

### BRIEF DESCRIPTION OF THE DRAWINGS

For a fuller understanding of the invention, reference should be made to the following detailed description, taken in connection with the accompanying drawings, in which:

FIG. 1 is an illustration showing the outline and cellular effects of ALA photodynamic therapy. The asterisk (*) indicates photoactivated protoporphyrin IX.

FIG. 2 is a graph showing the effect of viscosity on ALAS activity. The maximal rate of reaction decreases in direct proportion to solvent viscosity with a slope of one, indicating the reaction is diffusion controlled.

FIG. 3 is a pair of photographs showing the effect of ALAS activity on porphyrin production in E. coli. From left to right the ALAS variants harbor 0, 25, 100, 200, and 800 percent specific activity relative to the wild-type enzyme. Variants were grown to early stationary phase in LB media, and then equivalent aliquots were grown in MOPS overexpression buffer supplemented with glycine and succinate overnight, or spotted onto agar plates containing similar media.

FIG. 4 is a graph depicting flow cytometric analyses of porphyrin production in E. coli cells expressing differentially active ALAS variants. Cells were grown for 48 hours in MOPS buffer, and washed with 5 mM HEPES pH 7.2, 137 mM NaCl, and 2.7 mM KCl. prior to flow analysis.

FIG. 5 is an illustration of the overall outline of the methodology.

FIG. 6 is an illustration showing the strategy for generating and screening mutant libraries using error prone PCR.

FIG. 7 is an illustration showing Synthetic shuffling schematic for region I421-L438 of ALASs. X's denote non-conserved positions where degeneracy will be introduced, and y's indicate further incorporation of naturally ocurring diversity in supplemental oligonucleotides. The scale and number of oligos shown is for illustrative purposes only.

### DETAILED DESCRIPTION OF THE PREFERRED EMBODIMENT

The present invention provides an enhanced system of photodynamic therapy employing 5-aminolevulinate synthase (ALAS) variants. Photodynamic therapy (PDT) with 5-aminolevulinic acid is a promising approach to dysplastic disorders such as lung, esophageal, and bladder cancers, as well as atherosclerosis. PDT involves the local or systemic delivery of 5-aminolevulinate, which is then converted by natural cellular metabolism to the photosensitizing agent protoporphyrin IX. Upon light irradiation in the presence of dissolved oxygen, protoporphyrin IX catalyzes generation of reactive oxygen species and thereby stimulates apoptosis or necrosis in the light exposed cell. Although some cancers and plaque selectively accumulate protoporphyrin IX, the efficacy of photodynamic therapies in vitro can be dramatically enhanced by utilizing receptor mediated targeting to increase the accumulation of photosensitizer within the diseased cells.

The instant invention provides a means to achieve this goal leading to novel and effective treatment regimes. The approach involves targeting discrete cell populations with a 5-aminolevulinate synthase (ALAS) variant that has been specifically tailored, using directed evolution techniques, to stimulate optimal photosensitivity within the target cells. Specifically, it is proposed to generate genetically diverse ALAS cDNA libraries using error-prone PCR, DNA shuffling, DNA family shuffling, and synthetic shuffling. These libraries are subcloned into a cloning vector suitable for transformation of mammalian cells, and are then used to transform non-small cell cancer (NSCLC) cell lines. The transformed cells are screened by fluorescent activated cell sorting to identify and isolate the 1-2 variants that stimulate maximal protoporphyrin IX accumulation. The isolated ALAS variants can be targeted back to the NSCLC cells in a way that confers photosensitivity. Next, overexpression of the isolated variants in E. coli and covalently attaching epidermal growth factor to allow cellular uptake can be performed. The same cell line used to screen the variant libraries can then be exposed to these chimeric molecules and photosensitivity assessed. This general methodology will be applicable to a wide variety of cancers and proliferative disorders, resulting in patient-specific therapy.

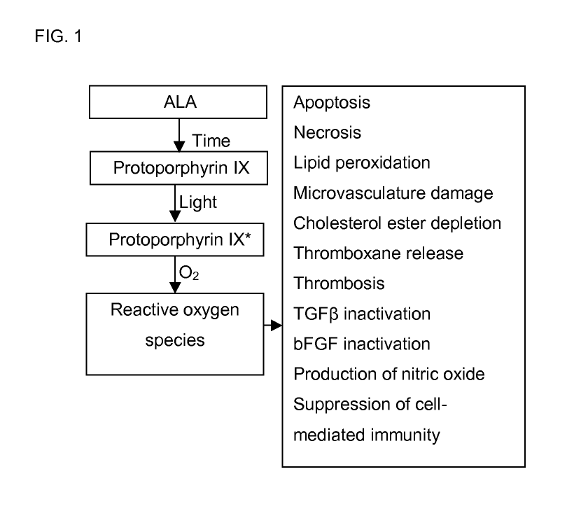

Photodynamic therapy (PDT) is an expanding area of research and clinical applications that utilize light activation of a photosensitizer in the presence of dissolved oxygen to generate reactive oxygen species and thereby stimulate apoptosis or necrosis in undesirable cells (MacDonald, I. J., et al., Journal of porphyrins and phthalocyanines (2001) 5:105-129.) The photosensitizer or photosensitizer precursor, such as 5-aminolevulinic acid (ALA; see FIG. 1), is delivered either topically or systemically and allowed from two to twenty-four hours to equilibrate within the target tissues. A light source is then applied (either externally or endoscopically depending upon the anatomical location of the target cells) specifically to the target tissue. The photosensitizer absorbs the light energy and preferentially transfers it to molecular oxygen to generate either singlet oxygen or superoxide radical anions, and it is primarily these reactive oxygen species that elicit the cytotoxic effects listed in FIG. 1. The effective area of oxidative action upon light exposure is limited to approximately 40 nanometers immediately surrounding the activated photosensitizer, which is less than 5% of the diameter of a typical mammalian cell (Kress, M., et al. J Biomed Opt (2003) 8:26-32; Takemura, et al. Photochem Photobiol (1989) 50:339-44), and along with precise light delivery this limited area of oxidative action provides a means of targeting specific cell populations for eradication while sparing healthy tissues. Therapeutic regimes for a variety of cancers caused by smoking have been developed, including lung cancers (Maziak, D. E., et al., Ann Thorac Surg (2004) 77:1484-91), esophageal cancer (Wolfsen, H. C. et al., Dig Dis (2002) 20:5-17), and bladder cancer (Jichlinski, P., et al. Urol Res (2001) 29:396-405), and applications in cardiovascular disorders appear equally promising (Kossodo, S., et al. Am J Cardiovasc Drugs (2001) 1:15-21). The advantages of PDT over other treatment modalities include relative non-invasiveness, accurate targeting, and excellent cosmetic outcomes. A major limitation is the lack of a means to provide adequately for selective accumulation of photosensitizer specifically within target tissues, with the result that repeated treatments are often required, many patients experiences severe systemic light sensitivity for up to a month following treatment, and therapeutic irradiation can unnecessarily damage healthy tissue surrounding the target tissue (Brown, S. B., et al., Lancet Oncol (2004) 5:497-508; Dougherty, T. J., et al. Photofrin. Lasers Surg Med (1990) 10:485-8).

In this regard recent effort has been directed towards developing the area of targeted PDT, which utilizes receptor-ligand interactions to drive selective accumulation of photosensitizer within discrete cell populations. (Sharman, W. M., et al., Adv Drug Deliv Rev (2004) 56:53-76; Demidova, T. N., et al. Int J Immunopathol Pharmacol (2004) 17:117-26; Vrouenraets, M. B., et al. Cancer Res (2001) 61:1970-5) It is expected that this approach will enhance efficacy by realizing higher target tissue photosensitizer concentrations, while potentially resulting in enhanced safety profiles due to the lower quantities of drug required to elicit a therapeutic response.

A variety of different photosensitizers suitable for PDT have been reported and efforts to create novel ones are ongoing. (Pushpan, S. K., et al. (2002) Curr Med Chem Anti-Canc Agents 2:187-207; Kelty, C. J., et al. (2002) Photochem Photobiol Sci 1:158-68; Detty, M. R., et al. (2004) J Med Chem 47:3897-915) Most of the photosensitizers reported so far fall into the two broad classes of porphyrins and porphyrin precursors based on ALA. The systemic or topical administration of ALA or ALA esters results in transient accumulation of protoporphyrin IX, which is a good photosensitizer with an excellent pharmacokinetic profile relative to most other photosensitizers. (Webber, J., et al. (1997) J Surg Res 68:31-7; Dalton, J. T., et al. (2002) J Pharmacol Exp Ther 301:507-12; Fukuda, H., et al. (2005) Int J Biochem Cell Biol 37:272-6) Protoporphyrin IX accumulates because ALAS is the rate-limiting enzyme of the porphyrin biosynthetic pathway. Protoporphyrin IX is not efficiently converted into heme (which is an ineffective photosensitizer) under these conditions presumably because porphyrin synthesis has been uncoupled from iron delivery by circumventing the natural regulation of ALAS activity. This results in the rate of porphyrin synthesis outpacing the rate of iron deliver to the enzyme ferrochelatase, which catalyzes conversion of protoporphyrin IX and iron into heme, and consequently protoporphyrin IX accumulates.

In a complicating twist, in vitro studies demonstrate that production of protoporphyrin IX from ALA is not only concentration dependent, but also cell line dependent (Tsai, T., R. et al. (2004) Lasers Surg Med 34:62-72; Chakrabarti, P., et al. (1998) Prostate 36:211-8; Bartosova, J., et al. (2000) Comp Biochem Physiol C Toxicol Pharmacol 126:245-52; Tsai, J. C., et al. (1999) Lasers Surg Med 24:296-305) The mechanisms behind the cell type specific response to a given concentration of ALA could include varying degrees of cellular ALA uptake, as well as varying levels of the enzymes subsequent to ALAS in the biosynthetic pathway to protoporphyrin IX. Furthermore, different cell types may also have different intrinsic photosensitivities due to differences in susceptibility to oxidative damage. These data suggest that although a targeting strategy might circumvent the issue of differential cellular ALA uptake, the maximal levels of protoporphyrin IX achievable may differ depending upon the cells targeted. This is a factor that could limit the applicability of ALA-PDT to only those cell types capable of producing sufficient protoporphyrin IX to induce cell death upon illumination. The research project proposed here offers a means to shed further light on these possibilities, and potentially address these cell type specific differences in a clinically relevant fashion by providing a means to deliver ALAS activity optimized to confer photosensitivity to a given cell type.

ALA is the precursor to all naturally occurring porphyrins, and is synthesized in animal mitochondria by the enzyme aminolevulinate synthase (ALAS) from the substrates glycine and succinyl-CoA. (Ferreira, G. C., et al. (1995) J Bioenerg Biomembr 27:151-9) ALAS occurs in animals and the alpha-subclass of purple bacteria, but not in plants or any other class of bacteria. Instead these organisms synthesize ALA from glutamate in three catalytic steps. (Reinbothe, S., et al. (1996) Eur J Biochem 237:323-43) Interestingly, two chromosomally distinct copies of functional ALAS are known to be present in many organisms, including mammals. The high degrees of sequence homology observed for the ALAS isoforms suggest gene duplication that arose as a means to allow for differential regulation of expression, and phylogenetic analyses support this hypothesis. In homo sapiens ALAS-1 (sometimes referred to as the “housekeeping” ALAS) is expressed in all tissues, with the highest levels occurring in the liver, where it appears to function in the production of heme for cytochromes. ALAS-2, on the other hand, is expressed almost exclusively in developing erythrocytes, where it supports heme synthesis for hemoglobin production. The erythroid specific ALAS contains an iron-responsive-element in the 5′-untranslated region that inversely coordinates iron availability with ALAS-2 mRNA translation, but this regulatory element is not present in the housekeeping ALAS mRNA (Cox, T. C., et al. (1991) Embo J 10:1891-902; Dandekar, T., et al. (1991) Embo J 10:1903-9) Mitochondrial import of mammalian ALAS isoforms is essential because the substrate succinyl-CoA is produced in the mitochondrial matrix, and both isoforms contain multiple heme regulatory motifs in the protein presequence that support heme feedback inhibition of mitochondrial import (Lathrop, J. T., et al. (1993) Science 259:522-5) It is possible to eliminate this feedback regulation by mutating the cysteine residues present in the heme regulatory motifs to alanine or serine, with the result that translocation into the mitochondrial matrix occurs even at elevated heme concentrations (Gagnebin, J., et al. (1999) Gene Ther 6:1742-50; Lathrop, J. T., et al. (1993) Science 259:522-5) Upon mitochondrial import the protein presequence is removed to form the mature ALAS enzyme, which appears to associate with the matrix side of the inner mitochondrial membrane. The erythroid specific ALAS may form a functional complex with succinyl-CoA synthetase, although such complexation is not absolutely required for catalytic activity, and the housekeeping ALAS does not complex succinyl-CoA synthetase. (Furuyama, K., et al. (2000) J Clin Invest 105:757-64) In summary, although expression of ALAS activity is highly regulated, these regulatory mechanisms are sufficiently understood to allow generation of viable ALAS constructs that can circumvent these regulatory mechanisms.

In the search to find new ways to increase quantum yield without stimulating prolonged systemic photosensitivity an innovative and productive approach might be to use evolving targeting technologies to selectively deliver enzymatic capacity to enhance endogenous production of ALA. Enhancing the specific activity of ALAS is considered essential to realize the optimal effectiveness of this approach because of the unusually low specific activity of this enzyme. The catalytic efficiency for the substrate glycine in particular, is only 3.4 mM⁻¹ min⁻¹ at 37 C and pH 7.2. (Tan, D., et al. (1998) Biochemistry 37:1478-84) for murine erythroid ALAS (k_{cat}/K_{M} for the other substrate, succinyl-CoA, is 20 μM⁻¹ min⁻¹ under similar conditions), meaning that even at glycine concentrations as high as 2 mM it still takes over a minute for one active site to produce the eight ALA molecules required to synthesize just one molecule of the photosensitizing agent protoporphyrin IX.

The in-vitro adenovirus delivery of an erythroid ALAS cDNA construct successfully modified to eliminate regulation by iron and heme has been described, and infection with this construct resulted in sufficient ALA production to partially stimulate PDT-mediated apoptosis of H1299 lung carcinoma cells. However, addition of desferrioxamine was necessary for an optimal response, and potentiated photosensitivity nearly two-fold. (Gagnebin, J., et al. (1999) Gene Ther 6:1742-50) Desferrioxamine is an iron chelator that inhibits conversion of protoporphyrin IX into heme Choudry, K., et al. (2003) Br J Dermatol 149:124-30), and the requirement for desferrioxamine in this study suggests that this approach did not result in a sufficient rate of ALA production to overwhelm the relatively slow rate of endogenous conversion of protoporphyrin IX to heme, and that better results might be obtained by increasing the ALAS activity delivered to the cells. The present invention provides a novel approach to PDT by constructing genetically diverse libraries of ALAS variants, and isolating individual variants from these libraries based on capacity to stimulate protoporphyrin IX production in the cells to be targeted for PDT. These variants will result in the selective accumulation of protoporphyrin IX in and immediately around a target cell population to a degree not previously possible, and will lead to improvements in the effectiveness of current PDT practice in treating a variety of diseases including tobacco related diseases.

5-Aminolevulinic acid is also known as 5-aminolaevulinic acid, 6-aminolevulinic acid, δ-aminolaevulinic acid and 5-amino-4-oxopentanoic acid. 5-Aminolevulinic acid can be used as the salt, particularly a simple salt and especially the hydrochloride salt. 5-Aminolevulinic acid can also be used in the form of a precursor or product of 5-aminolevulinic acid. 5-Aminolevulinic acid can also be used in its pharmacologically equivalent form, such as an amide or ester. Examples of precursors and products of 5-aminolevulinic acid and pharmacologically equivalent forms of 5-aminolevulinic acid that can be used in the present invention are described in J. Kloek et al., Prodruas of 5-Aminolevulinic Acid for Photodynamic Therapy, Photochemistry and Photobiology, Vol. 64 No. 6, December 1996, pages 994-1000; WO 95/07077; Q. Peng et al., Build-Up of Esterified Aminolevulinic-Acid-Derivative-Induced Porphyrin Fluorescence in Normal Mouse skin, Journal of Photochemistry and Photobiology B: Biology, Vol. 34, No. 1, June 1996; and WO 94/06424, which are all incorporated by reference herein in their entirety. As used herein, all of these compounds, unless other wise noted, are referred to jointly and severally as “ALA.”

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

The invention will be further described by way of the following non-limiting examples.

### EXAMPLE 1

Development and characterization of procedures for the creation, identification, and use of a 5-aminolevulinate synthase variant conferring maximal photosensitivity upon a given cell population.

ALAS is a pyridoxal phosphate dependent enzyme that is evolutionarily related to transaminases. A great deal of information regarding the structure and function of the enzyme has been generated by kinetic, spectroscopic, and point mutational analysis studies. Continuous steady-state kinetic assay for ALAS, which utilize cycling of the succinyl-CoA substrate to allow precise determination of ALA production rates at sub-saturating concentrations of this substrate down into the nanomolar range have been developed. (Hunter, G. A., et al. (1995) Anal Biochem 226:221-4) A structural and mechanistic model for the ALAS active site has been proposed based on sequence homologies and the known crystal structure and enzymology of the distantly related enzyme aspartate aminotransferase (Gong, J., et al. (1998) Biochemistry 37:3509-17), and point mutational analyses subsequently supported this model. (Tan, D., et al. (1998) Biochemistry 37:1478-84; Hunter, G. A., et al. (1999) Biochemistry 38:3711-8; Tan, D., et al. (1998) Protein Sci 7:1208-13) Based on the results of pre-steady state kinetic analyses of ALAS, a model wherein the enzyme exists in alternate “open” and “closed” conformations has been developed. (Hunter, G. A., et al. (1999) J Biol Chem 274:12222-8) It was postulated that the rate-limiting step controlling the production of ALA from glycine and succinyl-CoA is conversion of the “closed” conformation to the “open” conformation, which is coincident with release of ALA from the enzyme surface.

An ALAS crystal structure has not been reported, but the crystal structures of two very closely related enzymes have, and these structures shed further light on ALAS enzymology. ALAS is most closely related to a small family of pyridoxal phosphate dependent enzymes known as the alpha-oxoamine synthases, which catalyze condensation of a small amino acid with a Coenzyme A ester to form an aminoketone. The alpha-oxoamine synthase family is small but important, and includes ALAS, serine palmitoyltransferase (SPT), which catalyzes the initial and key regulatory step of sphingolipid biosynthesis (Hanada, K. (2003) Biochim Biophys Acta 1632:16-30), amino-oxononanoate synthase (AONS), which catalyzes the first committed step of biotin synthesis (Schneider, G., et al. (2001) FEBS Lett 495:7-11), and ketobutyrate ligase (KBL), which is involved in threonine catabolism and appears to be the only member of the family that catalyzes a readily reversible reaction. (Schmidt, et al. (2001) Biochemistry 40:5151-60) The crystal structure of both e. coli KBL and e. coli AONS have been solved, and for AONS both holoenzymic and product bound structures are available.

TABLE 1
Primary amino acid sequence homologies for E. coli.
AONS, E. coli KBL, and murine erythroid ALAS.

| | E. coli | meALAS | meALAS |
|---|---|---|---|
| | AONS | vs E. coli | vs E. coli. |
| Alignment | vs E. coli. | AONS | KBL |
| Identity | 31% | 29% | 31% |
| Strong | 23% | 23% | 22% |
| similarity | | | |
| Weak | 13% | 11% | 12% |
| similarity | | | |

The three dimensional alignment of the product bound crystal structures of E. coli AONS (Webster, S. P., et al. (2000) Biochemistry 39:516-28) and E. coli KBL. (Schmidt, et al. (2001) Biochemistry 40:5151-60) has been reported. Using the program DeepView (Schwede, T., et al. (2003) Nucleic Acids Res 31:3381-5), the backbone atoms of these structures are found to align with an overall three-dimensional RMS deviation of 1.23 angstroms. Coupled with the observation that Clustal W alignment of these two primary amino acid sequences with the catalytic core of murine erythroid ALAS indicate a similar degree of conservation amongst all three enzymes (TABLE 1), it is reasonable to conclude that the crystal structures of AONS and KBL are excellent models for the ALAS backbone quaternary structure to within an average of 1-2 angstroms. This provides a reasonable structural basis for rationalization of the mechanisms behind accrual of hyperactivity in ALAS variants, as well as providing a model for effective rational design experiments. For instance, substrate specificity of murine erythroid ALAS can be rationally adjusted by changing residue arginine 85, which is predicted to form a salt bridge with the carboxylate tail of the succinyl-CoA substrate, to leucine. This results in a higher catalytic efficiency for butyryl-CoA than succinyl-CoA, as would be expected (not shown). One would expect this change in substrate specificity to be even more dramatic in an R85L mutant also harboring a mutation of T430 to leucine or valine. This is because the side chain hydroxyl group of T430 is predicted to form a hydrogen bond with the carboxylate tail of succinyl-CoA. A three dimensional alignment of the holoenzymic and product bound AONS crystal structures indicates that while most of the structure is unchanged, an extended conformation “lid” near the C-terminus closes over the active site in the product bound structure. The authors concluded that the product was sterically precluded from dissociating from the enzyme surface until this lid relaxes to the “open” position (Webster, S. P., et al. (2000) Biochemistry 39:516-28; Alexeev, D., M. et al. (1998) J Mol Biol 284:401-19). These structural data provide good circumstantial evidence for the validity of the model proposed for ALAS catalysis, which was advanced prior to the solution of these crystal structures. (Hunter, G. A., et al. (1999) J Biol Chem 274:12222-8)

The ALAS catalytic core contains one conserved tryptophan residue, and based on the crystal structures of AONS and KBL, this residue resides at the base of the loop expected to undergo conformational changes during catalysis. It has been found that tryptophan fluorescent quenching of ALAS does occur upon ALA binding, and this can be observed and quantitated using our stopped-flow when the instrument is configured to maximize fluorescent sensitivity (data not shown). By determining the observed rate constants as a function of ALA concentration under pseudo first-order reaction conditions it may be possible to resolve the “close” and “open” rate constants for the domain movement associated with ALA binding and release. If the resolved rate constant for domain “opening” is comparable to k_{cat}, it would be strong evidence that the overall rate of catalysis is limited by relaxation of the active site “loop” or “lid” to an open conformation during ALA release.

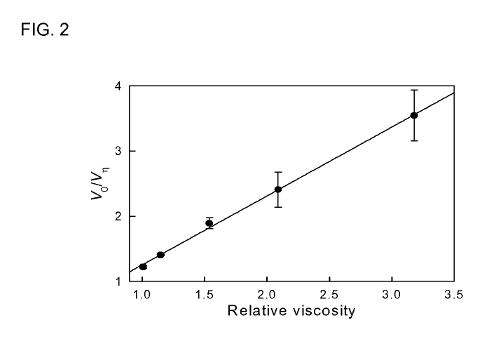

Further evidence that domain movement limits enzyme turnover comes from viscosity studies using glycerol as the viscogen (FIG. 2). A linear correlation was found to exist between the maximal rate of reaction and solvent viscosity. The best fit line gives a slope statistically indistinguishable from one, indicating the reaction is 100% diffusion limited.

While not intending to be bound by a particular theory, it is proposed that the ALAS region corresponding to the active site lid may be a hot spot for generation of enzyme hyperactivity. Amino acids I421-L438 of murine erythroid ALAS are centered in this region. Synthetic shuffling of these amino acid positions in order to obtain a library containing all possible combinations of the known naturally occurring amino acids in this region is one site of interest in the generation of ALAS variants (see below). Although not rational design per se, the observed data support the desirability of this strategy leading to a variety of hyperactive ALAS variants.

ALAS variants have been serendipitously created, but detailed characterization has not yet been reported in the literature. (Tan, D., et al. (1998) Biochemistry 37:1478-84; Gong, J., et al. (1995) Biochemistry 34:1678-85; Cheltsov, A. V., et al. (2001) J Biol Chem 276:19141-9) Interestingly, in every case observed so far the point mutant is either within or immediately adjacent to the C-terminal lid region considered to undergo a conformational change to allow release of ALA, except for one intriguing construct wherein two ALAS monomers are sequentially linked head to tail with a spacer between the catalytic cores to form a monomeric enzyme (2XALAS) with approximately 8-fold greater specific activity than the unlinked monomers. Even in this unusual case the hyperactivity observed could reasonably be attributed to strain induced by covalently linking the two monomers, possibly causing each monomer to preferentially shift towards a more open conformation in the ALA bound state wherein the rate of ALA release is accelerated.

It will be shown that that hyperactive ALAS variants can enhance transfected cancer cell line photosensitivity relative to the wild-type enzyme. pcDNA based constructs designed to facilitate mitochondrial import of the enzyme while eliminating regulation by iron or heme levels (Gagnebin, J., et al. (1999) Gene Ther 6:1742-50) are being constructed. These constructs will further the demonstration of effective ALAS variant. It is noted that 2XALAS is extremely unstable both thermally and proteolytically, and this could impact its capacity to stimulate protoporphyrin IX production relative to other ALAS variants in mammalian cells. The next most active variant, R433K ALAS, is only two-fold as active as the wild-type enzyme, which may affect its ability to significantly impact photosensitivity.

### Library Screening Strategies

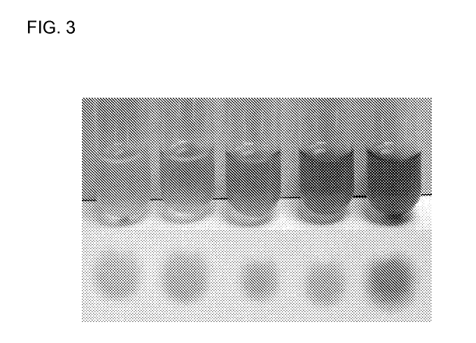

ALAS variant libraries expressed in E. coli can be screened based on fluorescent porphyrin production, as the expression of libraries in E. coli is straightforward, convenient, and relatively inexpensive. Porphyrin levels in E. coli cells overexpressing ALAS are proportional to ALAS variant activity, and these differences can be readily distinguished visually (FIG. 3), and quantitated spectrophotometrically (not shown). A recent study that included overexpression of Rhodobacter spheroides ALAS in E. coli and quantitation of resultant porphyrins using HPLC found that constitutive expression of ALAS alone led to a 40-fold increase in total porphyrins, while heme content was only increased 4-fold (Kwon, S. J., et al. (2003) Appl Environ Microbiol 69:4875-83).

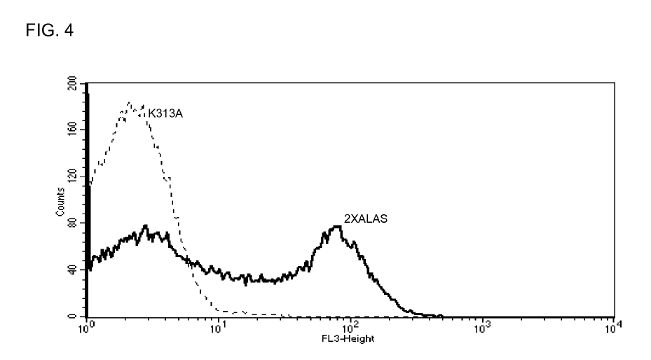

The porphyrin production induced by ALAS overexpression could be utilized for effective and convenient library screening with flow cytometry followed by fluorescent activated cell sorting (FACS) analyses. This would allow identification and isolation of variants expressing the highest levels of the fluorescent porphyrin protoporphyrin IX, which is also the porphyrin responsible for eliciting a photodynamic response. Flow cytometry experiments using overexpressed ALAS positive and negative controls is shown in FIG. 4, and this result has been confirmed in follow-up experiments at a 24 hour time point. The wild-type ALAS population is intermediate between K313A and 2XALAS, as expected (not shown). This approach appears to be well-suited to permit rapid high-throughput screening of libraries of up to and beyond 10⁶ variants.

One consideration to the expression and selection of ALAS variant libraries in E. coli is that stimulation of porphyrin biosynthesis in these cells may not necessarily correlate to similar effects in mammalian tumor cells. An ALAS variant that is effective at promoting photosensitivity in one tumor cell line may not be similarly effective in all other cell lines. Conceptually, the best cell line to express the variant library in would be as similar as possible to the cell or tissue type to eventually be targeted clinically, in order to best account for any possible background cellular metabolism that could affect variant ability to stimulate protoporphyrin IX accumulation. The use of FACS to successfully sort populations of both hematopoietic and lymphoblastoid cells based on protoporphyrin IX production have been reported, and expression systems for the high-throughput screening of libraries in mammalian cells are available (Fontanellas, A., et al. (1999) J Gene Med 1:322-30; Fontanellas, A., et al. (2001) Gene Ther 8:618-26; Laitinen, O. H., et al. (2005) Nucleic Acids Res 33:e42; Michiels, F., et al. (2002) Nat Biotechnol 20:1154-7). These considerations facilitate a strategy whereby the ALAS variant libraries are expressed in the tumor cell line to be targeted for PDT, and to isolate variants using FACS based on protoporphyrin IX production. Thus, mammalian cell expression and screening is one option for producing variants, while the expression and screening in E. coli can be used as an alternative approach.

### Research Design & Methods

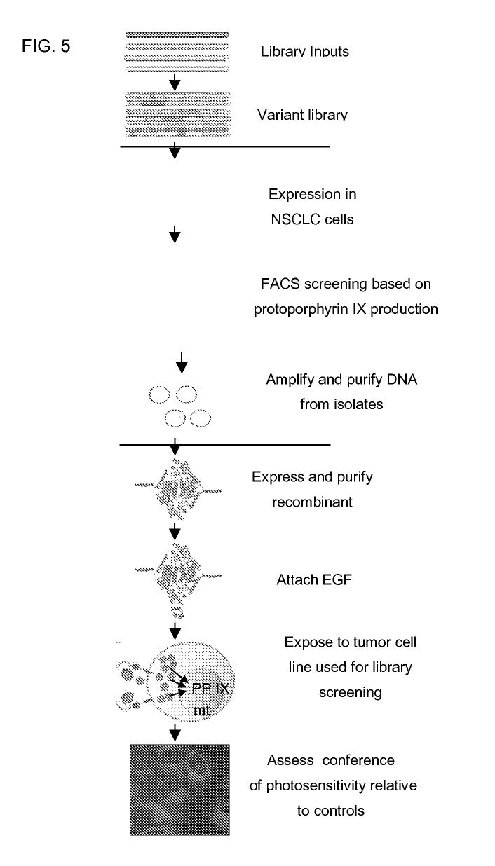

An outline of the methodologies is presented in FIG. 5. ALAS variant libraries are constructed using three different and complementary directed evolution strategies, in order to ensure a genetically diverse pool of variants likely to have useful attributes. Family based gene shuffling (Crameri, A., et al. (1998) Nature 391:288-91) is depicted for illustrative purposes. The cells to be targeted for PDT are transfected using a viral-based vector system that facilitates expression in both mammalian and bacterial cells (Laitinen, O. H., et al. (2005) Nucleic Acids Res 33:e42), and screened based on protoporphyrin IX production using FACS. The DNA from the most promising isolates is then purified. Finally, the DNA is transfected into E. coli and the overexpressed protein is purified and linked to EGF as has been reported (Gijsens, A., et al. (2000) Cancer Res 60:2197-202). These ALAS variant/EGF chimeras are then exposed to the same cell line used to conduct the library screening, and the photosensitivity relative to controls is assessed. These steps are discussed in more detail below.

### Construction of Alas Variant Libraries Using Error Prone PCR, DNA Family Shuffling, and Synthetic Shuffling.

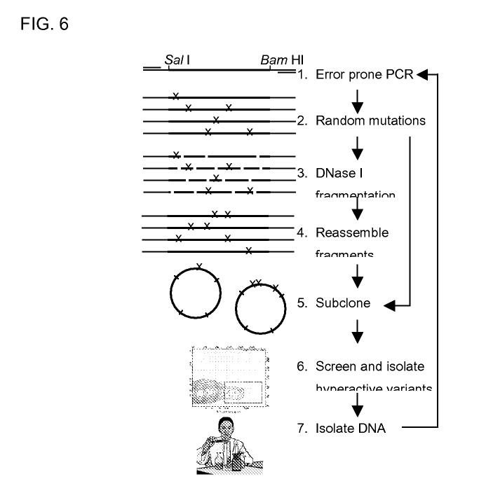

Maximizing genetic diversity will lead to hyperactive ALAS variants. To further the production of hyperactive ALAS variants three different and complementary engineering strategies can be used. The strategy for generating and screening mutant libraries using error prone PCR is illustrated in FIG. 6. An aim in the mutagenesis will be the introduction of an average of 1-4 new amino acid substitutions per library construct in each iteration of the overall process. This will ensure numerous active variants while avoiding a high frequency of unchanged sequence. The ALAS cDNA will be from pDT6, which encodes the mature murine erythroid ALAS cDNA with a five histidine tag at the amino terminus for simple and standardized purification (Ferreira, G. C., et al. (1993) J Biol Chem 268:584-90; Tan, D., et al. (1996) Biochemistry 35:8934-41). Error prone PCR will be conducted using Taq polymerase and a low purine/pyrimidine ratio, with adjustments to the MnCl₂ concentration to control mutation frequency within the range of 0.08-0.67%, which will facilitate achievement of the desired average mutation frequency per construct (Cadwell, R. C., et al. (1994) PCR Methods Appl 3:S136-40; Shafikhani, S., et al. (1997) Biotechniques 23:304-10).

Further expansion of mutant libraries can be achieved by DNA shuffling (also known as in vitro recombination), which involves limited fragmentation of the library with DNase I followed by PCR in the absence of primers, to essentially create a chimeric library of the mutant library (Stemmer, W. P. (1994) Proc Natl Acad Sci USA 91:10747-51). Both the mutant and recombinant mutant libraries can be subcloned into pBVboostFG (Laitinen, O. H., et al. (2005) Nucleic Acids Res 33:e42) or another suitable viral based vector, and screened for hyperactivity. The most promising 3-5 overall candidates can then be purified and quantitatively analyzed to assess the primary characteristics of enhanced catalytic efficiency, substrate specificity, and thermal/proteolytic stability. The best 1-2 variants can be selected and used for a subsequent round of PCR mutagenesis, and iterations of the entire process will continue until the catalytic efficiency for the substrate glycine is no longer enhanced at least two-fold, with no appreciable loss of substrate specificity or enzyme stability.

### Generation of DNA Family Shuffling Variant Libraries.

It has been reported that DNA shuffling of a family of highly homologous genes, such as homologs from different species, results in superior generation of functional diversity, as compared to error prone PCR (Crameri, A., et al. (1998) Nature 391:288-91). Naturally occurring genes are “pre-selected” for functional diversity, and millions to billions of years of evolution have resulted in a variety of favorable adaptations to a multiplicity of environments. To capitalize upon this natural occurring genetic diversity DNA family shuffling can be performed using ALAS genes from a variety of species. In one iteration these can be confined to mammalian ALASs', which should have relatively similar regulatory mechanisms at the protein level. In another iteration ALASs' with a more diverse evolutionary background can be shuffled and screened, but in this case it will only be the “catalytic core” of the enzymes that is shuffled, and the extended N- and C-termini observed in most eucaryotic ALASs' will not be included. Instead these regions can be filled in with the corresponding mature murine erythroid ALAS sequence. The ALAS clones used in these studies can be limited to those commercially available from A.T.C.C., or available from other researchers. This can provide an excellent cross-section of genetic diversity. Protocols for DNA family shuffling have been reported in the literature (Crameri, A., et al. (1998) Nature 391:288-91; Chang, C. C., et al. (1999) Nat Biotechnol 17:793-7).

Generation of a synthetically shuffled library for the ALAS region corresponding to the active site lid, the opening of which is required for product release and is considered to limit the overall rate of ALA production.

The goal of conducting synthetic shuffling specifically on the residues in the loop region of ALAS is to selectively enhance the genetic diversity in this region beyond what is expected to be accomplished by the error prone PCR and DNA family shuffling protocols, and to do this in a way that increases the probability of creating active variants with diverse properties.

As discussed above, multiple lines of evidence suggest that an extended conformation stretch of residues near the C-terminus of ALAS form a “loop” or “lid” that closes over the active site during catalysis and controls the rate of ALA production by limiting its release from the active site. The conformational change revealed in the holoenzymic and product bound crystal structures of AONS (Webster, S. P., et al. (2000) Biochemistry 39:516-28) corresponds to residues I421-L438 of mature murine erythroid ALAS (Ferreira, G. C., et al. (1993) J Biol Chem 268:584-90). It is most likely these residues that are involved in the conformational changes proposed to limit the rate at which ALA is produced, based on kinetic studies (Hunter, G. A., et al. (1999) J Biol Chem 274:12222-8).

Error prone PCR and DNA shuffling, although very powerful techniques, can be augmented using the technique of synthetic shuffling. The error prone PCR protocol generates more A:T and T:A interconversions than G:C and C:G interconversions and, due to the degeneracy of the genetic code, the probability of a particular position being mutagenized to any other amino acid does not follow a normal distribution (Shafikhani, S., et al. (1997) Biotechniques 23:304-10). On the other hand, DNA shuffling relies on recombination, which preferentially occurs in regions where sequence identity is high, with the result that the highly conserved regions such as murine erythroid ALAS I421-L438 are the regions most likely to recombine effectively and thus remain unchanged in progeny following shuffling. Synthetic shuffling, in comparison, allows deliberate construction of libraries containing all possible combinations of all the naturally occurring amino acids found in a gene family, such that the probability of creating an active construct with the unique qualities being screened for is dramatically increased (Ness, J. E., et al. (2002) Nat Biotechnol 20:1251-5).

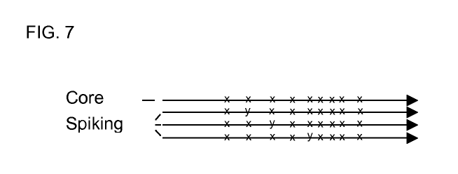

Synthetic shuffling can incorporate by design the random recombination of all of the naturally occurring amino acids corresponding to the murine erythroid ALAS I421-L438 region from at least 71 different ALAS sequences ranging from bacteria to humans into a minimal number of synthetic oligonucleotides. Diversity that can not be degenerately incorporated into the core oligonucleotide without also incorporating amino acids not found in nature can be added in supplemental oligonucleotides, as depicted in FIG. 7. It has been determined that eight different supplemental oligonucleotides will need to be synthesized in order to generate the 69, 120 possible unique variants in this region, using the 71 ALAS sequences known as of October 2004 as input. Following amplification the oligonucleotide library will be used as the internal primer for megaprimer mutagenesis (Sarkar, G., et al. (1990) Biotechniques 8:404-7).

### Identification and Isolation of Hyperactive Alas Variants Based on Stimulation of Protoporphyrin IX Production in NSCLC Cells.

Isolation of hyperactive ALAS variants by library expression in E. coli followed by FACS screening is one option. Due to differences in metabolic and regulatory landscapes moving from E. coli to human cells, expression and FACS screening of ALAS variant libraries in the same tumor cells that will later be exposed to the isolated variant and assessed for photosensitivity may prove most useful. This approach is expected to allow isolation of those variants most likely to prove effective in conferring photosensitivity to the target cells.

### Expression of Alas Variant Libraries in NSCLC Cells.

Library screening can be conducted using the recently reported multipurpose cloning vector pBVboostFG, which allows for the expression and screening of libraries in both mammalian and bacterial cells (Laitinen, O. H., et al. (2005) Nucleic Acids Res 33:e42). This cloning vector offers advantages with respect to other viral expression systems due to its versatility, simplicity, and lack of cytotoxicity; something that could negatively affect screening based on a phenotype of protoporphyrin IX production. In this regard an alpha-virus based expression system, while an option, may prove too cytotoxic (Koller, D., et al. (2001) Nat Biotechnol 19:851-5). An adenoviral system, the PhenoSelect platform, appears to be a suitable mammalian cell line expression system for library screening, and is one option to the pBVboostFG system (Michiels, F., et al. (2002) Nat Biotechnol 20:1154-7). NSCLC are advantageous cells for the methodologies because they encompass the majority of lung cancers, and are known to express EGFR; a common therapeutic target for NSCLC (Rosell, R., et al. 2004) Lung Cancer 46:135-48). Alternatively, the cell line chosen for could be one that is commercially available, or might be a new cell line derived from a patient tumor. In any case, the transformed cells chosen should be maintained in minimal light at all times.

### Construction of Functional ALAS-EGF Chimeras that are Internalized by NSCLC Cells and Transported into Mitochondria.

FACS screening and isolation of mammalian cells based on protoporphyrin IX production has been reported, as has the well-known induction of protoporphyrin IX biosynthesis in mammalian cells upon ALA exposure (Fontanellas, A., et al. (1999) J Gene Med 1:322-30; Fontanellas, A., et al. (2001) Gene Ther 8:618-26; Gamarra, F., et al. (2004) J Photochem Photobiol B 73:35-42; Brunner, H., et al. (2001) Photochem Photobiol 74:721-5; Bissonnette, et al. (2001) Photochem Photobiol 74:339-45; Luksiene, Z., et al. (2001) Cancer Lett 169:33-9; Stummer, W., et al. (1998) J Photochem Photobiol B 45:160-9; Steinbach, P., et al. (1995) Photochem Photobiol 62:887-95). FACS should permit rapid high-throughput screening of ALAS variant libraries as large as 10⁵-10⁶ cells.

cDNA isolation and sequencing of FACS selected variants, relying on common molecular biological techniques, can be performed. This will yield sequencing data that will hopefully prove to be of some general interest, as they will facilitate comparison of phenotypic changes with changes in protein structure.

### Re-Introduction of Isolated Variants into NSCLC Cells Using a Targeting System and Determination of Resultant Photosensitivity.

Once prospective variant cDNAs have been isolated they can be assayed for capacity to confer photosensitivity to NSCLC cells. This can be done by covalently linking EGF to the ALAS variant for intracellular delivery via EGFR-mediated endocytosis, as has been reported (Gijsens, A., et al. (2000) Cancer Res 60:2197-202). Other receptor mediated and viral delivery systems are also feasible.

### Overexpression of Selected Alas Variant Enzymes in e. coli, Followed by Purification.

The purified pBVboostFG plasmids, or other ALAS constructs, containing the ALAS cDNAs with the highest protoporphyrin IX accumulation activity can be transformed into E. coli strain DH5-α for overexpression and purification. Induction of overexpression can be initiated with IPTG and purification can rely on nickel chelate chromatography, as has been described (Tan, D., et al. (1998) Biochemistry 37:1478-84).

### Construction of Alas-EGF Chimeras that are Internalized by NSCLC Cells and Transported into Mitochondria.

Chimera construction will be carried out using glutaraldehyde as a cross-linking reagent as has been described previously (Gijsens, A., et al. (2000) Cancer Res 60:2197-202). Enzyme activity can be checked before and after exposure to glutaraldehyde to determine the optimal coupling conditions. Alternatively, the introduction of a strategically placed lysine residue into the constructs, use of an alternative cross-linking strategy, or switch to a baculovirus delivery system, for which pBVboostFG is well suited (Laitinen, O. H., et al. (2005) Nucleic Acids Res 33:e42), may be utilized. The possibility that glutaraldehyde cross-linking might interfere with enzyme activity is mentioned because the ALAS surface is anticipated to contain multiple basic residues in the succinyl-CoA binding site. Perhaps by conducting the cross-linking in the presence of succinyl-CoA these residues would be protected. Transport into mitochondria can be verified by Western blotting.

### Quantitative Assessment of the Photosensitivity Conferred by These Constructs, Relative to Free ALA.

Cells exposed to ALAS variants can be assessed for protoporphyrin IX levels and photosensitivity as described (Gagnebin, J., et al. (1999) Gene Ther 6:1742-50). Controls can include cells grown in the absence of light, and cells exposed to an inactive ALAS-EGF chimera, constructed by introducing a mutation at the active site lysine required for catalytic activity (Hunter, G. A., et al. (1999) Biochemistry 38:3711-8). Other controls and experimental parameters can be included as may become necessary or useful.

Additional techniques exist to enhance the genetic diversity of ALAS variant libraries, such as rational design of chimeras combining mutations identified as responsible for reaction rate acceleration, or DNA shuffling of a group of the most hyperactive variants. Further, if any particular position is found to have multiple amino acid substitutions in hyperactive variants the possibility of conducting saturation mutagenesis of that position to more fully evaluate its role in promoting hyperactivity can be considered. Another approach involves concatamerizing the most hyperactive variants, in analogy to the 2XALAS construct, to see if this results in further increases in catalytic efficiency. Directed evolution of the 2XALAS to enhance stability can also be pursued.

This invention provides a means to promote photosensitivity in a wide variety of disease cell phenotypes, including those found in tobacco related diseases. This project will demonstrate that it is possible to generate and screen ALAS libraries and isolate variants best suited for successful photosensitization of a particular cell type. The approach of screening using the target cells may find general applicability with other libraries that can couple a fluorescent signal with a desired phenotype. The general approach proposed here will be of interest in the treatment of other dysplasias, including atherosclerosis. Using cells from a patient's dysplasia for library screening, the treatment to be administered can be tailored specifically to the patients needs.

A variety of tobacco related dysplasias respond favorably to ALA-PDT modalities. The ability to selectively enhance ALA accumulation in target cells represents a significant further advancement in this area. Constructing and screening ALAS variants based on protoporphyrin IX production, using the same NSCLC cells to be targeted for therapy, will advance treatment of these diseases.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described above. In case of conflict, the present specification, including definitions, will control. In addition, the materials, methods, and examples are illustrative only and not intended to be limiting. The disclosure of all publications cited above are expressly incorporated herein by reference, each in its entirety, to the same extent as if each were incorporated by reference individually.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A method of producing a 5-aminolevulinate synthase (ALAS) variant having enhanced protoporphyrin IX accumulation in a target cell population comprising the steps of:
generating a cDNA library of ALAS variants;
providing a target cell population;
introducing in vitro the ALAS variants into the target cells;
screening the target cells for protoporphyrin IX accumulation; and
identifying the ALAS variants having enhanced protoporphyrin IX accumulation according to the results of screening.

2. The method according to claim 1 wherein the cDNA library of ALAS variants is generated using techniques selected from the group consisting of error-prone PCR, DNA shuffling, DNA family shuffling and synthetic shuffling.

3. The method according to claim 1 wherein the transformed target cells are screened for protoporphyrin IX accumulation by fluorescent activated cell sorting.

4. The method according to claim 1 wherein the target cell population is a non-small cell lung cancer cell line.

5. The method of claim 1 wherein the target cell population is a cell population of interest removed from a patient undergoing patient-specific therapy whereby the patient's cells are used to screen for ALAS variants having enhanced protoporphyrin IX accumulation in the patient's cell of interest.

6. The method of claim 5 wherein the patient is a cancer patient.

7. The method of claim 6 wherein the cancer is non-small cell lung cancer.

8. The method of claim 1 wherein the cDNA library of ALAS variants encode ALAS variant fusion proteins comprising a cellular uptake sequence adapted to enhance uptake of the ALAS variants into the target cell.

9. The method of claim 8 wherein the cellular uptake sequence is a sequence derived from epidermal growth factor.

10. A method of producing a 5-aminolevulinate synthase (ALAS) variant having enhanced activity in a target cell population comprising the steps of:
generating a cDNA library of ALAS variants;
providing a target cell population;
introducing in vitro the ALAS variants into the target cells;
screening the target cells for an indicia of ALAS activity; and
identifying the ALAS variants having enhanced ALAS activity according to the results of screening.

11. The method of claim 10 wherein the target cell population is a cell population of interest removed from a patient undergoing patient-specific therapy whereby the patient's cells are used to screen for ALAS variants having enhanced ALAS activity in the patient's cell of interest.

12. The method of claim 11 wherein the patient is a cancer patient.

13. The method of claim 12 wherein the cancer is non-small cell lung cancer.

14. The method of claim 10 wherein the cDNA library of ALAS variants encode ALAS variant fusion proteins comprising a cellular uptake sequence adapted to enhance uptake of the ALAS variants into the target cell.

15. A method for treating a target tissue in a subject with photodynamic therapy comprising the steps of:
administering to the subject an effective amount of 5-aminolevulinate synthase (ALAS), or an analog or variant thereof, or a nucleic acid encoding a 5-aminolevulinate synthase, or an analog or variant thereof;
irradiating the target tissue of the subject using radiation in an amount and of a wavelength effective to activate the 5-aminolevulinic acid variant, thereby treating a target tissue in the subject with photodynamic therapy.

16. The method according to claim 15 wherein the step of administering and the step of irradiating are separated by at least about 2 hours.

17. The method according to claim 15 wherein the step of administering and the step of irradiating are separated by about 2 hours to about 48 hours.

18. The method according to claim 15 wherein the step of administering and the step of irradiating are separated by about 2 hours to about 24 hours.

19. The method according to claim 15 further comprising the step of administering to the subject an effective amount of a porphyrin precursor.

20. The method according to claim 15 wherein the porphyrin precursor is 5-aminolevulinic acid.

21. The method according to claim 15 wherein the 5-aminolevulinate synthase is a fusion protein further comprising a cellular uptake sequence.

22. The method of claim 21 wherein the cellular uptake sequence is a sequence derived from epidermal growth factor.

23. The method of claim 15 further comprising the step of selecting the compound to be administered wherein the compound is selected based upon screening assay data demonstrating enhanced in vitro protoporphyrin IX accumulation in the target cell type following administration of the compound.

24. The method of claim 15 further comprising the steps of:
providing a target cell population;
introducing in vitro one or more ALAS variants into the target cells;
screening the target cells for protoporphyrin IX accumulation; and
identifying the ALAS variants having enhanced protoporphyrin IX accumulation according to the results of screening, wherein one or more ALAS variants identified will be used for administration to the patient.

25. The method according to claim 24 wherein the target cell population used for screening is a cell population derived from the target tissue of the subject to be treated.
